(19) United States
(12) Patent Application Publication
(10) Pub. No.: US 2004/0093068 A1
(43) Pub. Date: May 13, 2004

(54) Abdominal stent graft and delivery system

(76) Inventors: Lee C. Bergen, Arlington, MA (US); Michael Szycher, Lynnfield, MA (US)

Correspondence Address: PATENT GROUP, CHOATE, HALL & STEWART, EXCHANGE PLACE, 53 STATE STREET, BOSTON, MA, 02109, US

(21) Appl. No.: 10/351,287

(22) Filed: Jan. 23, 2003

Related U.S. Application Data
(60) Provisional application No. 60/398,187, filed on Jul. 24, 2002.

(51) Int. Cl. A61F 2/06

(52) U.S. Cl. 623/1.15

(57) ABSTRACT

A stent including a tubular metal frame having at least two ends and interior luminal and exterior non-luminal surfaces. The stent includes at least one fixation ring at each end of the frame non-rigidly connected to the frame by a plurality of fasteners. The fixation ring is a metal ring having a smooth zig-zag pattern and a plurality of spikes located on an exterior surface. The stent may be encapsulated by a smooth polymeric coating, and a textured polymeric coating may be disposed over the exterior non-luminal surface of the encapsulating stent.

### This application claims priority to Provisional Application No. 60/398,187, filed Jul. 24, 2002, the entire contents of which are incorporated herein by reference.

### FIELD OF THE INVENTION

This invention pertains to stent grafts for treatment of abdominal aortic and aortoiliac aneurysms and to delivery systems therefor.

### BACKGROUND OF THE INVENTION

The aorta delivers freshly oxygenated blood from the heart to the remainder of the body, with the exception of the lungs. After leaving the heart, the aorta travels through the chest to the abdomen, where it divides into the two iliac arteries. As a person ages, the aorta may become less elastic due to atherosclerosis or other factors. If the wall of the aorta is sufficiently weakened, it may dilate and bulge, forming an aneurysm.

Without treatment, the aneurysm will eventually rupture, causing dangerous bleeding into the abdomen. Only 35% of patients with burst abdominal aortic aneurysms survive.

However, traditional surgical techniques for repairing the aneurysm require invasive surgery in which a graft is used to replace the diseased portion of the aorta.

Endovascular repair of the aorta can be performed much less invasively by inserting a stent graft intraluminally to the diseased site via 1 or 2 small incisions. In general, an endovascular aortic repair device includes a fabric graft supported by a metallic stent or frame. These devices are deployed in a collapsed form and are either self-expandable or expanded by balloons. In many devices, the stent portion is equipped with hooks, barbs, and/or spikes for secure attachment to the artery. Other grafts employ high radial forces to seal the graft against the arterial wall. The graft fabric, commonly Dacron™ (DuPont) or PTFE, is normally positioned inside the supporting stent and is secured over the hooks and spikes of the metallic frame. Biological materials, for example, fibrin, hydrolyzable gelatin, or collagen, are frequently included in these devices to seal the fabric.

These stent grafts have several failure modes. Some failures are related to defects in the metal, such as those caused by corrosion, which result in fracture of the support and attachment structure of the stent. In addition, defects in the fabric or detachment of the fabric from the stent may lead to failure. There may also be leakage either directly through the fabric or by failure of the seal between the stent and the arterial wall. In addition, the use of hooks, barbs, and balloons may cause additional injury to the already weakened artery. Successful endovascular repair requires exclusion of the aneurysm from circulation, maintenance of flow through the aorta and iliac arteries, and prevention of blood leaks into the aneurysm.

### SUMMARY OF THE INVENTION

In one aspect, the invention is a stent graft including a tubular metal stent having a first end, at least a second end, and interior luminal and exterior non-luminal surfaces. A smooth polymeric coating encapsulates the metal stent, and a textured polymeric coating is disposed over the exterior non-luminal surface of the encapsulated stent. The polymeric coating may include a non-biodegradable, biocompatible polyurethane.

The metal stent may include a first mesh cylinder having first and second ends and first and second fixation rings non-rigidly connected to the first and second ends of the first mesh cylinder, respectively bio-plurality of fasteners. Each fixation ring is a metal ring having a smooth zig-zag pattern. A plurality of spikes are located on an exterior surface of the fixation rings and penetrate though the textured polymeric coating.

The stent graft may deliver an anti-proliferative agent from its end. The antiproliferative agent may be Rapamycin, a Rapamycin analog, Taxol, or a Taxol analog. The stent graft may provide a coagulant at at least a portion of its exterior surface. The coagulant may be covalently or non-covalently linked to the exterior surface of the stent graft. The coagulant may be released from the exterior surface of the stent graft over a pre-determined period of time, for example 24 or 48 hours or less. The coagulant may be thrombin. The textured polymeric coating may be adapted and constructed to mechanically interlock with a blood clot and may have a texture of a tangle of threads. The graft may deliver a anti-thrombolytic agent from at least a portion of the interior luminal surface.

In another aspect, the invention is a stent including a tubular metal frame having a first end, at least a second end, and interior luminal and exterior non-luminal surfaces. The stent has a fixation ring at each end of the frame non-rigidly connected to the frame by a plurality of fasteners. The fixation ring is a metal ring having a smooth zig-zag pattern, and a plurality of spikes are disposed on an exterior surface of the fixation rings.

The metal frame may include a first mesh cylinder having first and second ends and first and second fixation rings non-rigidly connected to the first and second ends of the first mesh cylinder, respectively. The stent may further include second and third mesh cylinders each having first and second ends and third, fourth, fifth, and sixth fixation rings non-rigidly connected to the first and second ends of the third of the second and third mesh cylinders, respectively. The first ends of the second and third mesh cylinders are linked to the second and of the first mesh cylinder by a non-rigid mechanical connection between the second fixation ring and the third and fifth fixation rings. The stent may further include seventh, eighth, ninth, and tenth fixation rings disposed between the third, fourth, fifth, and sixth fixation rings and the second and third mesh cylinders, respectively, wherein adjacent rings are non-rigidly connected to one another and to the adjacent mesh cylinder by a member of fasteners, sutures, and both. Additional fixation rings may be disposed between the first and second fixation rings, respectively and the first mesh cylinder. These additional fixation rings are non-rigidly connected to the adjacent fixation ring and the first mesh cylinder by a member of fasteners, sutures, and both.

The first fixation ring of the metal frame may each comprise a plurality of eyelets adapted and constructed to receive the fasteners. The fasteners may be selected from metal loops, wire, and suture. The spikes may have a pyramidal shape. The spikes may be between 0.5 mm and 3 mm tall; for example, they may have a height of 2 mm and a base of at least 1 mm².

In another aspect, the invention is a delivery and positioning system for an abdominal aneurysm repair device comprising a delivery device. The delivery device includes an inflatable over-the-wire delivery catheter, a first cover adapted and constructed to be disposed over exterior surfaces of a abdominal aneurysm repair device disposed over the delivery catheter, and a second cover adapted and constructed to contain the covered abdominal aneurysm repair device within a single package. A radiopaque marker may be disposed on the second cover of the delivery device. The first cover may include a pair of longitudinal seams along which the cover is adapted and constructed to be separated into two portions. The delivery and positioning system may further include an over-the-wire positioning catheter having a radiopaque portion and a gripper. The positioning catheter delivers the gripper to the deliver device.

In another aspect, the invention is a method of delivering an abdominal aneurysm repair device. The method includes the steps of providing an abdominal aneurysm repair device mounted in a delivery system, inserting the delivery device into a desired location in an abdominal artery exhibiting an aneurysm in a patient, removing the second cover from the abdominal aneurysm repair device into first and second portions along an axis approximately parallel to the delivery catheter and removing first portion from the abdominal aneurysm repair device, removing the second portion of the first cover from the abdominal aneurysm repair device, directing the abdominal aneurysm repair device against an adjacent arterial wall, and removing the delivery catheter and the guidewire from the patient. The step of directing may include inflating the delivery catheter or increasing the temperature of a fluid circulating through the delivery catheter.

In another aspect, the invention is a method of delivering a bifurcated abdominal aneurysm repair device. The method includes providing an abdominal aneurysm repair device mounted in a delivery system, inserting the delivery device over the guidewire into a desired location and a first artery in a patient, inserting the positioning catheter over a second guidewire to mate with the delivery device at a predetermined location on the delivery device, removing the second cover from the abdominal aneurysm repair device, manipulating one arm of the abdominal aneurysm repair device into a second artery in the patient, wherein the first and second arteries are in fluidic communication, separating the first cover into first and second portions along an axis approximately parallel to the first artery and removing the first portion from the abdominal aneurysm repair device, removing the second portion of the first cover from the abdominal aneurysm repair device, directing the abdominal aneurysm repair device against an adjacent arterior wall, and removing the delivery and positioning catheters and the guidewires from the patient.

In another aspect, the invention is a method of making an abdominal aneurysm repair device. The method comprises the steps of providing a first cylindrical metal mesh having first and second ends, and non-rigidly attaching at least one fastening ring to each of the first and second ends. The fastening ring is a metal ring having a smooth zigzag pattern and a plurality of spikes disposed on its exterior surface. The method may further comprise providing at least second and third mesh cylinders having a smaller cross-sectional area than the first metal mesh, non-rigidly attaching at least one fastening ring to each end of each of the second and third mesh cylinders, and attaching the fastening ring at one end of the first metal mesh to the fastening rings at the ends of the second and third mesh cylinders. For example, two fastening rings may be attached at each end of the first, second, and/or third mesh cylinders.

The mesh cylinder may be encapsulated in a non-biodegradable, biocompatible polyurethane. A textured coating on an exterior surface of the mesh cylinder, wherein the spikes penetrate through the textured coating. The metal may be a shape memory alloy or a stress induced memory alloy.

### BRIEF DESCRIPTION OF THE DRAWING

The invention is described with reference to the several figures of the drawing, in which,

FIG. 1A is a schematic view of a straight stent according to an embodiment of the invention;

FIG. 1B is a schematic view of a bifurcated stent according to an embodiment of the invention;

FIG. 2 is a closer view of an end of the stent depicted in FIGS. 1A and 1B;

FIG. 3 is a schematic view of the surface of a fixation ring for use with an embodiment of the invention;

FIG. 4 is a schematic view of a straight stent graft according to an embodiment of the invention;

FIG. 5 is a series of schematic diagrams illustrating the deployment of a stent graft according to an embodiment of the invention; and

FIG. 6 is a schematic view of a patient receiving a stent graft according to an embodiment of the invention.

### DETAILED DESCRIPTION OF CERTAIN PREFERRED EMBODIMENTS

The invention provides a stent graft for repair of an abdominal aortic aneurysm. The stent graft includes a tubular metal stent having a first end and at least a second end. As used herein, the term “tubular” includes a structure having an elongated hollow shell and includes both branched structures and straight cylinders. A fixation ring is non-rigidly connected to each end of the stent by a plurality of fasteners. The fixation ring is a metal ring in a smooth (wavy) zig-zag shape with a plurality of pyramidal spikes protruding from an exterior surface thereof. A smooth polymeric coating completely encapsulates the metal stent and the fixation rings, and a textured polymeric coating is disposed over the exterior, non-luminal surfaces. The pyramidal spikes on the fixation rings penetrate through the polymeric coating.

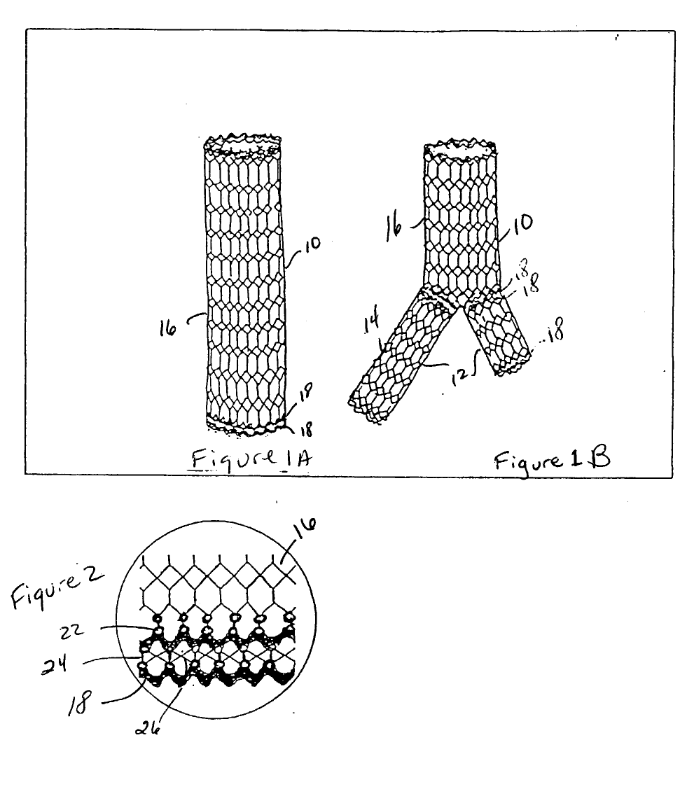

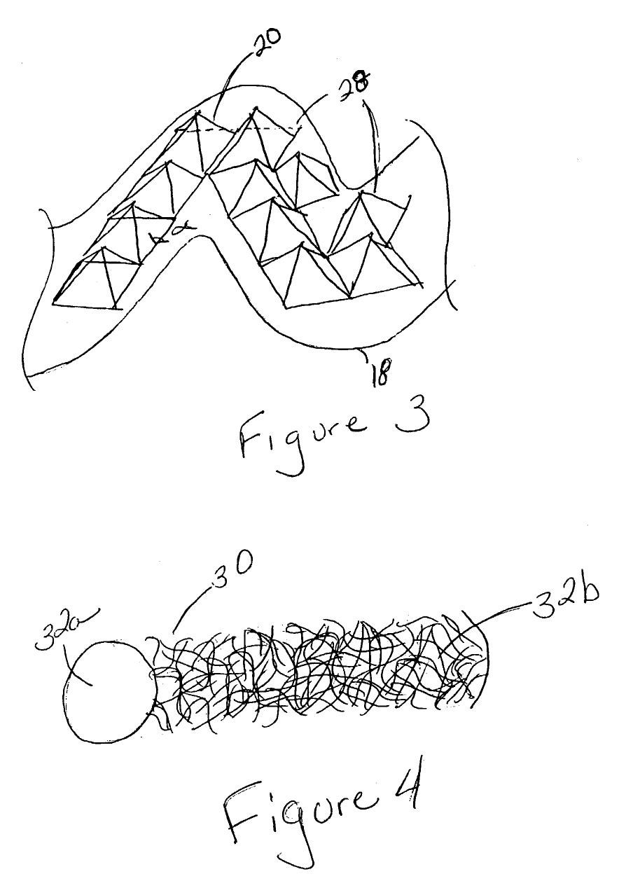

FIG. 1 shows exemplary straight and bifurcated stents for an endovascular abdominal aortic aneurysm repair according to an embodiment of the invention. Stent 10 is shown with a basic hexagonal-square mesh pattern. Alternative mesh patterns known to those skilled in the art may be exploited as well. Some examples may be found in U.S. Pat. Nos. 6,059,822, 5,968,070, 5,178,618, and 5,824,053. The stent 10 may include a single cylinder (FIG. 1A) or may be bifurcated into branches 12 (FIG. 1B). The branches 12 are produced by attaching smaller diameter mesh cylinders 14 to a larger mesh cylinder 16 using fixation rings 18 as described below.

The mesh cylinders 16 and 14 may be produced from a TiNi shape memory alloy (e.g., Nitinol) and are typically between 1 and 2.5 cm in diameter and about 10 to 16 cm long. Stress induced memory alloys, such as those described in U.S. Pat. Nos. 5,067,957 and 4,505,767, may also be employed. The stent may be produced from woven wire, a wire mesh with welded joints, or rolled and welded foil, or cut from a tube. One skilled in the art will realize that stents may be produced in various sizes and with various lengths to accommodate patients of various sizes.

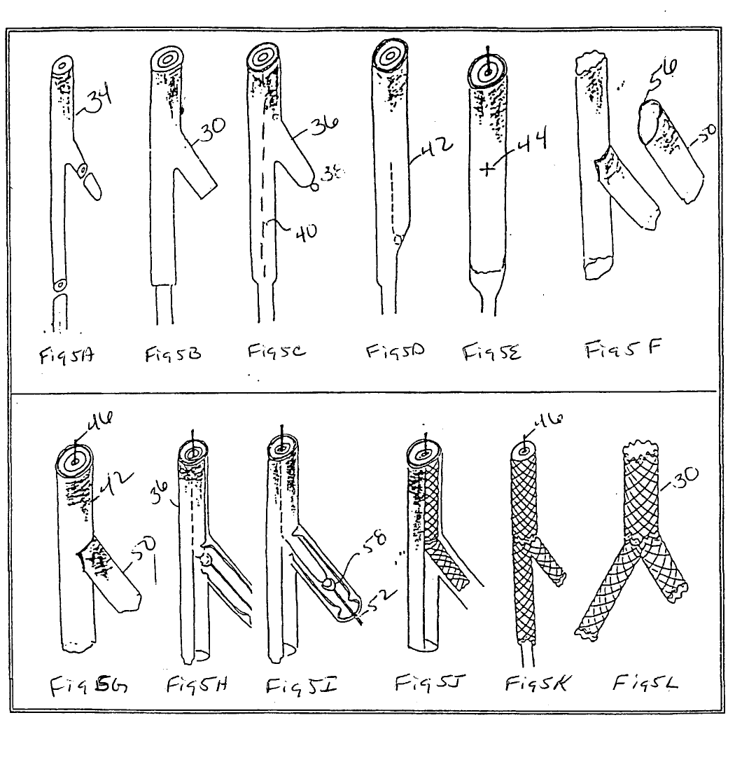

Each end of stent 10 has one, two, or more fixation rings 18 at each end. FIG. 2 provides a closer view of the attachment of fixation rings 18 to one another and to mesh cylinders 16 and 14. Each fixation ring 18 is a thin ring in a smooth zig-zag shape having a external surface texture including a multitude of pyramidal spikes 20 (FIG. 3). The apexes of the fixation rings 18 include eyelets 22 for attachment of the fixation rings to one another and to mesh cylinder 16. For example, the fixation rings may be connected to one another using a combination of fasteners 24 and sutures 26. Fasteners 24 may be metal loops, shaped wire, or suture threaded through eyelets 22. The same fasteners may be used to attach branches 12 to mesh cylinder 16 via fixation rings 18 to form a bifurcated stent. The use of non-rigid fasteners that allow the fixation rings 18 and the mesh cylinders 16 and 14 to move with respect to one another facilitates flexing of the stent 10 under the peristaltic (pulsed) pressure of blood flowing through the aorta.

When the endovascular device of the invention is deployed, spikes 20 (FIG. 3) help attach the device to the surrounding arterial wall. The spikes are typically between 0.5 and 3 mm high, preferably at least 1.5 to 2 mm high. The pyramidal shape is achieved by direct cutting of the metal substrate (e.g., with a laser), which provides stability for the spikes and prevents them from collapsing onto the surface of the fixation rings 18. The angle α of the walls 28 and the area density of the pyramids are dependent on a variety of factors, including the manufacturing method, the design of the stent, thickness of the fixation rings, the hardness of the metal, the cutting equipment (e.g., laser type), requirements for deburring, losses in electropolishing, etc. The density of the spikes 20 also depends on their height, since taller spikes will likely have a larger footprint on the fixation ring 18. In one embodiment, a spike 2 mm high has a base at least 1 mm in area. One skilled in the art will be able to optimize the spike density and height with respect to manufacturing, design, and other requirements.

The stent 10 is completely encapsulated in a smooth, non-biodegradable, biocompatible polymer. An exemplary polymer for use with the invention is ChronoFlex™, a polycarbonate-polyurethane co-polymer with internal polysiloxane segments from CardioTech International (see U.S. Pat. No. 5,863,627, the entire contents of which are incorporated herein by reference). One skilled in the art will recognize that a variety of biocompatible, non-biodegradable polymers, including polyurethanes, may be used to coat the stent 10. Preferably, the polymer has an elasticity similar to normal veins or arteries and is resistant to stress cracking and other mechanical and chemical degeneration in vivo.

The stent 10 may be encapsulated with the polymer using standard techniques, including but not limited to electro-spraying, painting, or dipping the stent 10 into a liquid polymer or prepolymer and allowing the polymer to set. The polymer serves several structural functions in the stent graft. It encapsulates the mesh cylinders 14 and 16 and fixation rings 18, including eyelets 22, fasteners 24, and sutures 26. Encapsulation prevents direct contact between blood and the stent and leaching of nickel from the stent into the blood, which would weaken the metal and eventually cause fracture. The coating additionally reduces friction between the stent graft and flowing blood. It also reinforces the overlapping wires if a woven wire is used to fabricate the stent. The polymer coating may also be made thick enough to prevent blood from leaking through the wall of the stent graft 28 into the aneurysm.

Stent graft straight 30, including a straight embodiment of stent 10, internal polymer coating 32a and external polymer coating 32b, is shown in FIG. 4. The coating 32a on the inside of the stent graft 30 is impermeable by blood and smooth to prevent turbulence. The external coating 32b may be textured to promote clot adhesion to the outside of stent graft straight 30. For example, the texture may resemble a tangle of threads with variable porosity. Other geometrical textures that promote clot fixation, e.g., woven or knitted, may be employed as well.

The polymer may also provide a medium for delivering various drugs and bioactive agents to the tissues at specific portions of the stent graft. For example, a suitable pharmacological agent may be eluted at the ends of the stent graft to prevent endothelialization, inflammation, and infection at the ends of the stent graft. Exemplary anti-proliferative agents include Rapamycin (available as Sirolimus from Wyeth-Ayerst), Taxol, and analogs of these, which are well known to those skilled in the art.

In one embodiment, the portion of the outer surface coating of the stent graft situated centrally in the aneurysm elutes a coagulant such as thrombin. The coagulant is released within a relatively short time after deployment, preferably within one or two days, and facilitates clot formation of the blood within the aneurysm. In a preferred embodiment, the coagulant is immobilized on the outside of the stent graft. Without liquid blood, the aneurysm will not pulsate in response to blood flow through the device, reducing stress on the arterial wall. The clotting blood also adheres to the textured coating on the outer surface of stent graft 30, further contributing to fixation of the device within the blood vessel.

In some embodiments, it may be desirable to have the inside luminal coating 32a of the stent graft 30 elute an anti-coagulant such as heparin, preferably low molecular weight heparin. The use of the stent graft to deliver the anti-coagulant guarantees that it will be delivered to a desired site over a period of time to supplement systemically administered anticoagulant, which is distributed throughout the body and is not focused at a specific target site.

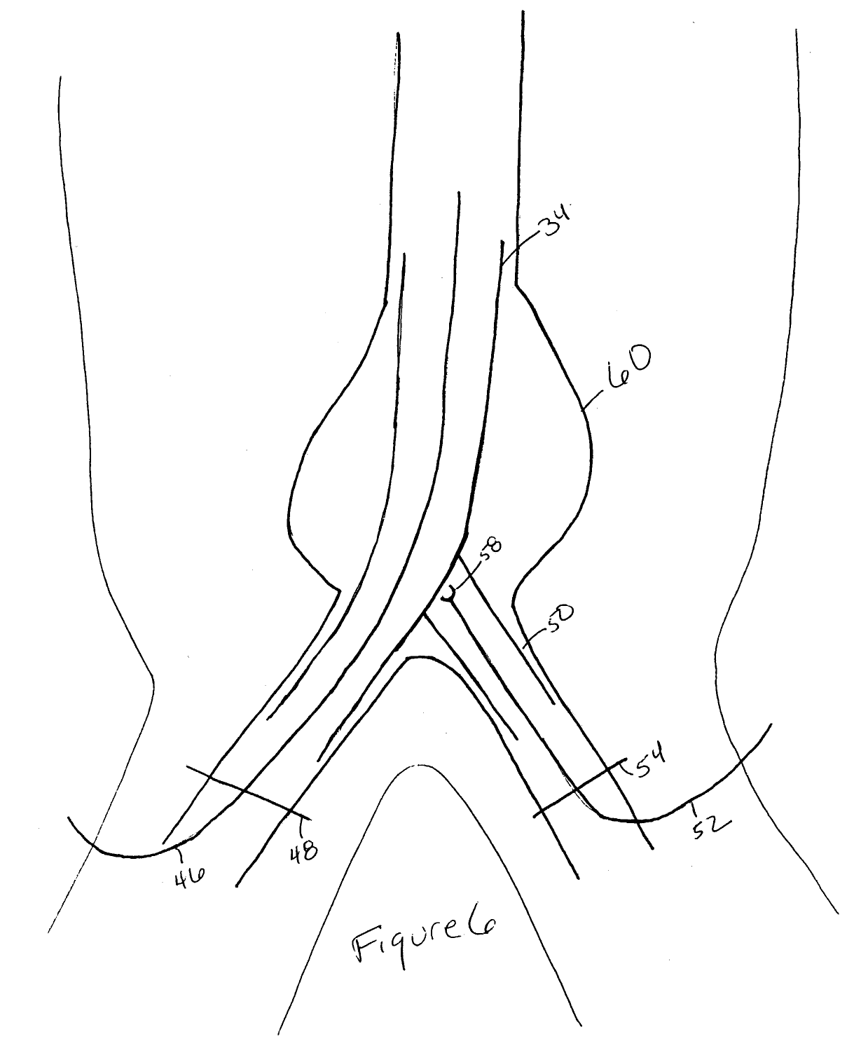

The stent graft 30 is fabricated as a unitary device so that it may be positioned within the aorta using a single delivery catheter. Bifurcated stent grafts require a second incision to guide the second branch of the device into position but are still delivered as a monolithic device using only one delivery catheter. A delivery system for bifurcated stent graft 30 is assembled using an over-the-wire delivery catheter 34 (FIG. 5A). Delivery catheter 34 is a cylindrical shell balloon. The stent graft 30 is mounted over delivery catheter 34 (FIG. 5B). The stent graft 30 should be collapsed to facilitate delivery. Delivery catheter 34 may be formed from a variety of bio-compatible polymers, including, for example, silicone (e.g., Silastic, from Dow Corning), polyurethanes (e.g., Hydrocoat, from Access Technologies), polytetrafluoroethylene, elastomeric hydrogels (e.g., Aquavene, from Menlo Care), and low density polyethylene. One skilled in the art will recognize other materials that may be used for the catheter. The stiffness of the material should be optimized to reduce thromogenicity while maintaining ease of insertion.

A cover 36 is disposed over the stent graft (FIG. 5C). The cover 36 surrounds the entire outer surface of the stent graft. A small ring 38 or other type of attachment is provided at the end of the free stent graft leg to provide a grip as the device is manipulated into place. Cover 36 is separable at a longitudinal seam 40. A second cover 42 is disposed around cover 36 (FIG. 5D). Cover 42 does not include an arm for the branch of a bifurcated stent graft but rather wraps the entire stent graft 30 into a single compact package. Cover 42 includes a radiopaque marker 44 to aid proper placement of the delivery device (FIG. 5E).

To deploy the stent graft 30, the delivery catheter 34 with mounted stent graft 30 and covers 36 and 42 is inserted over a guide wire 46 through an incision 48 in the groin area through an iliac artery into the abdominal aorta (FIG. 6). If the stent graft 30 is bifurcated, a positioning catheter 50 is inserted over a guide wire 52 through a second incision 54 in the groin area, accessing the other iliac artery (FIG. 5F, FIG. 6). Positioning catheter 50 preferably includes a lip 56 coated with a radiopaque material, preferably gold or platinum. The delivery catheter 34 and positioning catheter 50 are properly aligned with the aid of a fluoroscope or other probe (FIGS. 5G, 6). Once the delivery and positioning catheters (FIG. 5D) are in place, cover 42 is removed and a gripper 58, for example, a biopsy forceps, is inserted through the positioning catheter 50 (FIGS. 5H, 6). The gripper 58 is used to grab the ring 38, and bring one branch 12 of the stent graft into positioning catheter 50, thereby extending the device into the second iliac artery (FIGS. 5I, 6).

After the stent graft 30 has been manipulated into position, cover 36 is separated at the seam 40 by slight inflation of the end portion of the delivery catheter 34 and one of the halves is removed (FIG. 5J). Delivery catheter 34 has two walls defining a central cavity bounded by a hollow shell; increasing fluid pressure within the shell causes the shell to expand outwards. The second half of cover 36 is also removed and the stent graft 30 deployed by fully inflating the balloon delivery catheter 34 (FIG. 5K). If a shape memory nitinol alloy is used in the stent graft 30, then cold fluid should be circulated in the delivery catheter 34 as the stent graft 30 is positioned within the artery. The cold fluid is then replaced with warm fluid to allow the alloy to transform into the final shape. One skilled in the art will realize that the exact temperatures of the cold and warm fluids will depend on the composition of the alloy used in stent 10. Expansion of the stent graft 30 against the artery inserts spikes 20 into the arterial walls, immediately fixing the stent graft 30 in place and sealing aneurysm 60. The gripper 58, delivery catheter 34, positioning catheter 50, and the guide wires 46 and 52 are removed (FIG. 5L), and the incision or incisions are closed.

One skilled in the art will recognize that the delivery system described above may be used to deliver other straight and bifurcated stents, stent grafts, and other abdominal aneurysm repair devices having designs other than those taught herein. Where a bifurcated stent is delivered in two pieces rather than a monolithic unit, positioning catheter 50 should be replaced with a second delivery catheter 34. Additional radiopaque markers may be used to ease attachment of the two pieces within the patient.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A stent graft, comprising:
a tubular metal stent having a first end and at least a second end and interior luminal and exterior non-luminal surfaces;
a smooth polymeric coating encapsulating the metal stent; and
a textured polymeric coating disposed over the exterior non-luminal surface of the encapsulated stent.

2. The stent graft of claim 1, wherein the metal stent comprises:
a first mesh cylinder having first and second ends;
first and second fixation rings non-rigidly connected to the first and second ends of the first mesh cylinder, respectively, by a plurality of fasteners, wherein each fixation ring is a metal ring having a smooth zigzag pattern;
a plurality of spikes located on an exterior surface of the fixation rings, wherein the spikes penetrate through the textured polymeric coating.

3. The stent graft of claim 2, wherein the spikes have a pyramidal shape.

4. The stent graft of claim 2, wherein the spikes are between 0.5 mm and 3 mm tall.

5. The stent graft of claim 4, wherein the spikes have a height of 2 mm and a base of at least 1 mm².

6. The stent graft of claim 1, wherein the polymeric coatings comprise a non-biodegradable, biocompatible polyurethane.

7. The stent graft of claim 6, wherein the polyurethane is CHRONOFLEX™.

8. The stent graft of claim 1, wherein the stent graft delivers an anti-proliferative agent from its ends.

9. The stent graft of claim 8, wherein the anti-proliferative agent is selected from Rapamycin, a Rapamycin analog, Taxol, and a Taxol analog.

10. The stent graft of claim 1, wherein the stent graft provides a coagulant at at least a portion of its exterior surface.

11. The stent graft of claim 10, wherein the coagulant is covalently or non-covalently linked to the exterior surface of the stent graft.

12. The stent graft of claim 10, wherein the coagulant is released from the exterior surface of the stent graft over a pre-determined period of time.

13. The stent graft of claim 12, wherein the predetermined period of time is less than 48 hrs.

14. The stent graft of claim 13, wherein the predetermined period of time is less than 24 hrs.

15. The stent graft of claim 10, wherein the coagulant is thrombin.

16. The stent graft of claim 1, wherein the textured polymeric coating is adapted and constructed to mechanically interlock with a blood clot.

17. The stent graft of claim 1, wherein the textured polymeric coating has a texture of a tangle of threads.

18. The stent graft of claim 1, wherein the graft delivers an anti-thrombolytic agent from at least a portion of the interior luminal surface.

19. A stent, comprising:
a tubular metal frame having a first end and at least a second end and interior luminal and exterior non-luminal surfaces;
a first fixation ring at each end of the frame and non-rigidly connected to the frame by a plurality of fasteners, wherein the fixation ring is a metal ring having a smooth zigzag pattern; and
a plurality of spikes located on an exterior surface of the fixation rings.

20. The stent of claim 19, wherein the metal frame comprises:
a first mesh cylinder having first and second ends; and
first and second fixation rings non-rigidly connected to the first and second ends of the first mesh cylinder, respectively.

21. The stent of claim 20, further comprising:
second and third mesh cylinders each having first and second ends; and
third, fourth, fifth, and sixth fixation rings non-rigidly connected to the first and second ends of the second and third mesh cylinders, respectively, wherein the first ends of the second and third mesh cylinders are linked to the second end of the first mesh cylinder by a non-rigid mechanical connection between the second fixation ring and the third and fifth fixation rings.

22. The stent of claim 21, further comprising seventh, eighth, ninth, and tenth fixation rings disposed between the third, fourth, fifth, and sixth fixation rings, respectively, and the second and third mesh cylinders, respectively, wherein adjacent fixation rings are non-rigidly connected to one another and to the adjacent mesh cylinder by a member of fasteners, sutures, and both.

23. The stent of claim 20, further comprising third and fourth fixation rings disposed between the first and second fixation rings, respectively, and the first mesh cylinder, wherein the third and fourth fixation rings are non-rigidly connected to the first and second fixation rings, respectively, and the first mesh cylinder by a member of fasteners, sutures, and both.

24. The stent of claim 19, wherein the first fixation ring and the metal frame each comprise a plurality of eyelets adapted and constructed to receive the fasteners.

25. The stent of claim 19, wherein the fasteners are selected from metal loops, wire, and suture.

26. The stent of claim 19, wherein the spikes have a pyramidal shape.

27. The stent of claim 19, wherein the spikes are between 0.5 mm and 3 mm tall.

28. The stent of claim 19, wherein the spikes have a height of 2 mm and a base of at least 1 mm².

29. A method of making an abdominal aneurysm repair device, comprising:
providing a first cylindrical metal mesh having first and second ends; and
non-rigidly attaching at least one fastening ring to each of the first and second ends,
wherein the fastening ring is a metal ring having a smooth zigzag pattern and a plurality of spikes disposed on its exterior surface.

30. The method of claim 29, further comprising:
providing at least second and third mesh cylinders having a smaller cross-sectional area than the first metal mesh;
non-rigidly attaching at least one fastening ring to each end of each of the second and third mesh cylinders; and
attaching the fastening ring at one end of the first metal mesh to the fastening rings at the ends of the second and third mesh cylinders.

31. The method of claim 30, wherein non-rigidly attaching includes attaching two fastening rings at each end of the second and third mesh cylinders.

32. The method of claim 29, wherein non-rigidly attaching includes attaching two fastening rings at each end of the first mesh cylinder.

33. The method of claim 29, further comprising encapsulating the mesh cylinder in a non-biodegradable, biocompatible polyurethane.

34. The method of claim 33, wherein the polyurethane is CHRONOFLEX™.

35. The method of claim 33, further comprising depositing a textured coating on an exterior surface of the mesh cylinder, wherein the spikes penetrate through the textured coating.

36. The method of claim 29, wherein the metal is a shape memory alloy.

37. The method of claim 29, wherein the metal is a stress induced memory alloy.
